(19) United States
(12) Patent Application Publication
(10) Pub. No.: US 2024/0316110 A1
(43) Pub. Date: Sep. 26, 2024

(54) COMPOSITION COMPRISING HUMAN TONSIL STEM CELL-DERIVED ENDOPLASMIC RETICULUM AS ACTIVE INGREDIENT

(71) Applicant: PLCOSKIN Co., Ltd., Seoul (KR)

(72) Inventors: Woo Yeol Baek, Seoul (KR); Kyu Sik SHIM, Seoul (KR); Do Hyun KIM, Seoul (KR)

(73) Assignee: PLCOSKIN Co., Ltd., Seoul (KR)

(21) Appl. No.: 18/694,793

(22) PCT Filed: Nov. 30, 2021
(86) PCT No.: PCT/KR2021/017840

§ 371 Date: Mar. 22, 2024

(30) Foreign Application Priority Data
Sep. 24, 2021 (KR) 10-2021-0126659

(51) Int. Cl. A61K 35/28; A61K 8/14; A61K 8/98; A61P 17/00; A61Q 19/08; C12N 5/0775

(52) U.S. Cl. CPC A61K 35/28; A61K 8/14; A61K 8/981; A61P 17/00; A61Q 19/08; C12N 5/0668

(57) ABSTRACT

The present invention relates to a composition containing vesicles derived from human tonsil-derived stem cells as an active ingredient. The composition has the effects of promoting the expression of skin regeneration- and antioxidant-related genes in senescence-induced fibroblasts, and inhibiting fibroblast senescence.

### TECHNICAL FIELD

The present invention relates to a composition, particularly a composition for skin regeneration and anti-aging, containing vesicles derived from human tonsil-derived stem cells as an active ingredient.

### BACKGROUND ART

Aging is characterized by time-dependent loss of function and regenerative properties of organisms. Factors related to skin aging cause damage to cells, delaying skin regeneration and cell proliferation, cellular senescence. Cellular senescence is characterized by an irreversible arrest of the cell cycle and alteration of the focal adhesive cytoskeleton.

Cellular senescence in skin tissues is induced by various factors, such as oxidative stress, mitochondrial dysfunction, and ultraviolet irradiation.

For the past several decades, many researchers have been conducting research to overcome cellular senescence.

Recently, to overcome cellular senescence, many studies have focused on the tissue regenerative potential of exosomes.

Exosomes, which are known as nano-size biomimetic nanovesicles secreted across the plasma membrane from the originated cells by the endocytic pathway, contain several components, including miRNA, mRNA, and proteins. In addition, exosomes have been studied for skin rejuvenation and antiaging approaches.

However, despite the potential exosomes for therapeutic purposes, there are several hurdles, including low efficiency, long procedure time, and high technical expertise.

To overcome those hurdles, many researchers have focused on the direct production of exosome-mimetic nanovesicles from somatic cells. These biomimetic nanovesicles can be directly isolated from the desired cells by sonication and/or extrusion and have been reported to share similar characteristics with exosomes. Given the similar properties, cell-derived biomimetic nanovesicles could be utilized for drug delivery, tissue regeneration, and cancer targeting. In particular, it has been reported that nanovesicles derived from human tonsil-derived mesenchymal stem cells attenuate liver fibrosis and inflammation. In addition, an anticancer pharmaceutical composition containing nanovesicles derived from human tonsil-derived stem cells is known.

### PRIOR ART DOCUMENTS

### Patent Documents

Korean Patent Application Publication No. 10-2020-0141868

### DISCLOSURE

### Technical Problem

An object of the present invention is to provide a composition for skin regeneration and anti-aging prevention containing vesicles derived from human tonsil-derived stem cells.

### Technical Solution

In accordance with one aspect of the present invention, there is provided a composition for skin regeneration and skin anti-aging containing vesicles derived from human tonsil-derived stem cells as an active ingredient.

### Advantageous Effects

The composition according to one embodiment of the present invention has skin regeneration and anti-aging effects and may be efficiently produced.

### BRIEF DESCRIPTION OF DRAWINGS

FIG. 1a show is the morphology of human tonsil-derived mesenchymal stem cells produced in Preparation Example 1 (scale bar=200 μm).

FIG. 1b shows the expression of surface markers of human tonsil-derived mesenchymal stem cells produced in Preparation Example 1.

FIG. 2a shows protein expression and SEM images of nanovesicles produced from human tonsil-derived mesenchymal stem cells (TMSC-NVs).

FIG. 2b shows the results of dynamic light scattering analysis of nanovesicles produced from the human tonsil mesenchymal stem cells produced in Preparation Example 1.

FIG. 3 relates to the regulation of proliferation and senescence by treatment with TMSC-NVs in a passage-associated senescence model. Specifically, FIG. 3a shows morphological changes in human dermal fibroblasts (HDFs) (scale bar=200 μm), FIG. 3b shows the proliferation of passage-associated senescent HDFs after treatment with TMSC-NVs, FIG. 3c shows the results of senescence-associated β-galactosidase assay (scale bar=200 μm), FIG. 3d shows the quantitative analysis of SA-β-galactosidase assay, FIG. 3e shows the expression of vinculin in focal adhesion of HDFs, and 3f shows quantitative data of vinculin expression in focal adhesion.

FIG. 4 relates to the regulation of extracellular matrix and antioxidant genes in HDFs by treatment by TMSC-NVs in a passage-associated senescence model. Specifically, FIG. 4a shows mRNA expression of COL1, ELASTIN, SOD2, and HMOX1 in passage-associated senescent HDFs, FIG. 4b shows the results of immunofluorescence analysis of collagen type 1 in passage-associated senescent aged HDFs; and FIG. 4c shows quantitative data of immunofluorescence analysis.

FIG. 5 relates to the regulation of proliferation and senescence by treatment with TMSC-NVs in a UV-induced senescence model. Specifically, FIG. 5a shows morphological changes in HDFs by treatment with TMSC-NVs (scale bar=200 μm), FIG. 5b shows a proliferation test for UV-induced HDFs after treatment with TMSC-NVS, FIG. 5c shows SA-β-galactosidase assay of UV-induced senescent HDFs after treatment with TMSC-NVs (scale bar=200 μm), FIG. 5d shows quantitative data of the SA-δ-galactosidase assay, FIG. 5e shows the expression of vinculin in focal adhesion of HDFs, and FIG. 5f shows quantitative data of vinculin expression in focal adhesion.

FIG. 6 relates to the regulation of extracellular matrix and antioxidant genes by treatment with TMSC-NVs in a UV-induced senescence model. Specifically, FIG. 6a shows mRNA expression of COL1, ELASTIN, SOD2, and HMOX1 in UV-induced senescent HDFs, 6b FIG. shows the results of immunofluorescence analysis of collagen type 1 in UV-induced senescent HDFs, and FIG. 6c shows quantitative data of immunofluorescence analysis.

FIG. 7 relates to the regulation of proliferation and senescence by treatment with CD146+ TMSC-NVs in a passage-associated senescence model. Specifically, FIG. 7a shows the mRNA expression of COL1 and HMOX1 in senescent HDFs, FIG. 7b is shows the results of senescence-associated β-galactosidase assay (scale bar=200 μm), and FIG. 7c shows the quantitative analysis of SA-β-galactosidase assay,

FIG. 8 relates to the results of immunostaining after treatment with CD146+ TMSC-NVs in a skin aging model obtained by irradiating human skin tissue with ultraviolet B (UVB). Specifically, FIG. 8a shows the results of immunostaining for collagen type 1, collagen type 3, involucrin, and filaggrin, and FIG. 8b shows quantitative data of the immunostaining results.

### BEST MODE

Hereinafter, the present invention will be described in more detail.

The composition for skin regeneration and anti-aging according to one aspect of the present invention contains vesicles derived from human tonsil-derived stem cells as an active ingredient.

In the present specification, the term “stem cells” refers to undifferentiated cells capable of self-renewal and differentiation into two or more different types of cells.

In the present specification, the term “active ingredient” refers to an ingredient that can exhibit the desired activity alone or in combination with a carrier, which is not active by itself.

In the present specification, the term “nanovesicles” refers to nano-sized vesicles which are obtained from adult stem cells and have a nano-size similar to that of exosomes, which are extracellular vesicles.

In addition, nanovesicles are composed of phospholipid bilayers, which are the basic structure of biological membranes and separate the inside of the cells from the outside. Nanovesicles can not only contain water-soluble molecules (including DNA) or drugs therein, but also fat-soluble drugs can be attached thereto or positively and negatively charged substances can be bound thereto. Phospholipids are amphipathic in nature and have a molecular structure containing an anionic or zwitterionic polar group and two nonpolar fat-soluble chains with various degrees of unsaturation of about 16 hydrocarbons, and thus when phospholipids are dispersed in water, they spontaneously form vesicles.

In the field of applied science, nanovesicles are used in the cosmetics industry and in drug delivery and as a model for delivering genetic material to cells being cultured in vitro. Currently, it is possible to entrap both water-soluble and fat-soluble substances in nanovesicles, it is easy to target specific tissues using nanovesicles, it is easy to the size and modify nanovesicles, and nanovesicles have almost no toxicity problems due to the use of phospholipids, and can entrap more drugs than other drug carriers.

In the present specification, the term “vesicles” mainly refers to extracellular vesicles, and the term “extracellular vesicles” may refer to vesicles which are surrounded by a lipid bilayer and released into the external environment by all types of cells.

Extracellular vesicles may be called by various names, such as exosomes, microvesicles, ectosomes, microparticles, membrane vesicles, nanovesicles, and outer membrane vesicles, based on their origin, secretion mechanism, size, etc.

The vesicles may be one type selected from among extracellular vesicles, microvesicles, and nanovesicles. In particular, the vesicles may be nanovesicles.

According to one embodiment of the present invention, the nanovesicles derived from human tonsil-derived stem cells express cell surface markers that are specifically expressed in exosomes.

According to one embodiment of the present invention, the human tonsil-derived stem cells may be human tonsil-derived mesenchymal stem cells, without being limited thereto.

According to another embodiment of the present invention, the human tonsil-derived stem cells may be CD146 positive.

The nanovesicles may have a diameter of 50 nm to 250 nm, or 30 nm to 200 nm.

More specifically, the nanovesicles may have a diameter between 30 nm, 32 nm, 34 nm, 36 nm, 38 nm, 40 nm, 42 nm, 44 nm, 46 nm, 48 nm or 50 nm and 200 nm, 190 nm, 180 nm, 170 nm, 160 nm, 150 nm, 140 nm, 130 nm, 120 nm, 110 nm, 100 nm, 95 nm, 90 nm, 85 nm, 80 nm, 75 nm or 70 nm. For example, the nanovesicles may have a diameter of 30 to 100 nm, 40 to 80 nm, 50 to 100 nm, or 50 to 80 nm.

According to one embodiment, the nanovesicles may have a diameter of 40 to 55 nm. More specifically, the nanovesicles may have an average diameter between 40 nm, 42 nm, 44 nm, 46 nm, 48 nm or 50 nm and 55 nm, 54 nm, 53 nm, 52 nm, 51 nm or 50 nm. For example, the nanovesicles may have an average diameter of 42 to 53 nm, 46 to 52 nm, 48 to 52 nm, or 50 nm.

According to one embodiment of the present invention, the vesicles may further contain at least one immune antigen selected from among CD14, CD34, CD45, CD73, CD90, and CD146.

According to one embodiment of the present invention, the human tonsil-derived stem cells may be produced by a method comprising steps of: digesting human tonsil tissue with collagenase type 1 and DNase 1; filtering and centrifuging the digested product and removing the supernatant to obtain a cell pellet; and culturing the cells obtained from the cell pellet to obtain human tonsil-derived stem cells. In one embodiment, the step of digesting the human tonsil tissue with collagenase type 1 and DNase 1 may be performed in low-glucose Dulbecco's modified Eagle's medium (DMEM). The step of culturing the cells obtained from the cell pellet may be performed in DEME containing 10% fetal bovine serum, antibiotics, and antimycotics.

The nanovesicles may be produced by a method comprising steps of: suspending subcultured human tonsil-derived mesenchymal stem cells in a culture medium, followed by centrifugation and removing the supernatant; and resuspending the cell pellet from which the supernatant has been removed, and then passing the cells sequentially through two or more filters with different pore sizes using an extruder.

The two or more filters with different pore sizes may be used in the order from filters with large pore sizes to filters with small pore sizes. For example, the two or more filters with different pore sizes may include a filter with a pore size of 8 to 12 μm, a filter with a pore size of 3 to 7 μm, and a filter with a pore size of 0.2 to 0.6 μm. For example, the two or more filters with different pore sizes may be used in the order of filters with pore sizes of 10 μm, 5 μm, and 0.4 μm.

Meanwhile, the method of obtaining CD146-positive vesicles may further comprise steps of: treating the obtained human tonsil-derived stem cells with an FcR blocking reagent, and then treating the cells with CD146 microbeads, followed by incubation; treating the incubated cells with a magnetic-activated cell sorting (MACS) buffer, followed by centrifugation and removing the supernatant; and separating CD146-positive and CD146-negative cells by a MACS separator and a column, for example, an LS column. According to one embodiment, the step of treating the cells with CD246 microbeads, followed by incubation, may be performed under light-shielded conditions.

Alternatively, the method of obtaining CD146-positive vesicles may further comprise steps of: treating and incubating the obtained human tonsil-derived stem cells with an anti-CD146 antibody or a fluorophore-conjugated anti-CD146 antibody; and separating CD146-positive and CD146-negative using flow cytometry, after the incubation. Alternatively, the method may further comprising a step of separating CD146-positive and CD146-negative cells by capturing the obtained human tonsil-derived stem cells onto a surface to which an anti-CD146 antibody has been introduced. The surface to which the anti-CD146 antibody has been may be any surface to which the antibody can attach, and specific examples thereof include plastic plates, metal plates, metal alloy plates, polymer nanoparticles, metal nanoparticles, and the like.

In addition, CD146-positive nanovesicles derived from human tonsil-derived stem cells may be produced by a method comprising a step of producing CD146-positive human tonsil stem cell-derived nanovesicles from cells selected by a CD146 cell surface marker. Here, the step of producing nanovesicles may be performed according to the same method as the above-described method of producing nanovesicles from human tonsil-derived stem cells.

According to one embodiment, the composition for skin regeneration and anti-aging may be a pharmaceutical composition or a cosmetic composition.

According to one embodiment, the composition may be a pharmaceutical composition.

In addition to the vesicles, the pharmaceutical composition may further contain pharmaceutical adjuvants, such as preservatives, stabilizers, wetting agents or emulsifying agents, salts for regulating the osmotic pressure and/or buffers, and other therapeutically useful substances, and may be formulated in oral or parenteral dosage forms according to conventional methods.

The oral dosage forms include, for example, tablets, pills, hard and soft capsules, solutions, suspensions, emulsions, syrups, powders, fine granules, granules, pellets, and the like, and these dosage forms may contain, in addition to the active ingredient, surfactants, diluents (e.g., lactose, dextrose, sucrose, mannitol, sorbitol, cellulose, and glycine), and lubricants (e.g., silica, talc, stearic acid and its magnesium or calcium salts, and polyethylene glycol). Tablets may also contain binders such as magnesium aluminum silicate, starch paste, gelatin, tragacanth, methylcellulose, sodium carboxymethylcellulose, and polyvinylpyrrolidine, and may optionally contain pharmaceutical additives, including disintegrants such as starch, agar, alginic acid or its sodium salt, absorbents, colorants, flavoring agents, and sweeteners. The tablets may be prepared according to conventional mixing, granulating or coating methods.

In addition, the parenteral dosage forms may be transdermal dosage forms, including, for example, but not limited to, injections, drops, ointments, lotions, gels, creams, sprays, suspensions, emulsions, suppositories, and patches.

The pharmaceutical composition of the present invention may be prepared in a unit dose form or prepared to be contained in a multi-dose container by formulating with pharmaceutically acceptable carriers and/or excipients, according to a method that a person skilled in the art can easily perform. In this case, the dosage form of the pharmaceutical composition may be a solution, suspension or emulsion in oil or aqueous medium, or an extract, powder, granule, tablet or capsule, and may further contain a dispersing agent or a stabilizer.

Pharmaceutically acceptable carriers that may be contained in the pharmaceutical composition of the present invention include, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil, which are commonly used for formulation. In addition to the above ingredients, the pharmaceutical composition of the present invention may further contain lubricants, wetting agents, sweeteners, flavoring agents, emulsifiers, suspending agents, preservatives, etc. Suitable pharmaceutically acceptable carriers and formulations are described in detail in Remington's Pharmaceutical Sciences (19th ed., 1995).

The pharmaceutical composition of the present invention may be administered orally and parenterally, for example, by intravenous injection, subcutaneous injection, intramuscular injection, intraperitoneal injection, topical administration, intranasal administration, intrapulmonary administration, intrarectal administration, intrathecal administration, ocular administration, dermal administration, and transdermal administration.

The appropriate dosage of the pharmaceutical composition of the present invention may vary depending on factors such as formulation method, administration mode, patient's age, weight, sex, pathological condition, diet, administration time, administration route, excretion rate, and reaction sensitivity, and an ordinarily skilled physician can readily determine and prescribe the effective dose for desired treatment or prevention.

Determination of the dosage of the active ingredient is within the level of those skilled in the art. Although the daily dosage of the drug varies depending on various factors such as the degree of progression, the onset time, age and health condition, and complications in the subject to be administered the drug, the composition may be administered one to three times a day at a dose of 1 μg/day to 200 mg/kg for an adult in one embodiment, and 50 μg/kg to 50 mg/kg for an adult in another embodiment. The dose is not intended to limit the scope of the present invention in any way.

According to one embodiment of the present invention, the composition may be a cosmetic composition. For example, the cosmetic composition may be formulated into a solution, suspension, emulsion, paste, gel, cream, lotion, powder, soap, surfactant-containing cleanser, oil, powder foundation, emulsion foundation, wax foundation, leave-on type formulation, mist, spray, and the like, without being limited thereto. More specifically, the cosmetic composition may be formulated into detergents such as shampoos, conditioners, or body cleansers; hair styling agents such as hair tonics, gels or mousses; hair cosmetic compositions such as hair nourishing lotions, hair essences, hair serum scalp treatments, hair treatments, hair conditioners, hair shampoos, hair lotions, hair tonics, or hair dyes; and basic cosmetics such as oil-in-water (O/W) type or water-in-oil (O/W) type.

In addition, each formulation of the composition may contain, in addition to the above-mentioned active ingredient, other ingredients that may be appropriately selected by those skilled in the art without difficulty depending on the type or intended use of other external preparations. For example, it may further contain sunscreen, hair conditioning agent, fragrance, etc.

The cosmetic composition may contain a cosmetically acceptable medium or base. The cosmetic composition may be provided in the form of any formulation suitable for topical application. For example, the cosmetic composition may be provided in the form of a solution, a gel, a solid or pasty anhydrous product, an emulsion obtained by dispersing an oil phase in a water phase, a suspension, a microemulsion, a microcapsule, a microgranule, or an ionic (liposomal) or non-ionic vesicle dispersion, or in the form of cream, skin softener, lotion, powder, ointment, spray, or conceal stick. These compositions may be prepared according to conventional methods known in the art.

If the formulation of the present invention is a solution or emulsion, a solvent, a solubilizer, or an emulsifier may be used as a carrier ingredient. For example, the carrier ingredient may be water, ethanol, isopropanol, ethyl carbonate, ethyl acetate, benzyl alcohol, benzyl benzoate, propylene glycol, 1,3-butylglycol oil, glycerol aliphatic ester, polyethylene glycol, or fatty acid ester of sorbitan.

If the formulation of the present invention is a suspension, a carrier ingredient used may be a liquid diluent such as water, ethanol, or propylene glycol, a suspending agent such as ethoxylated isostearyl alcohol, polyoxyethylene sorbitol ester, or polyoxyethylene sorbitan ester, microcrystalline cellulose, aluminum metahydroxide, bentonite, agar, tragacanth, or the like.

If the formulation of the present invention is a paste, cream, or gel, a carrier ingredient used may be animal oil, vegetable oil, wax, paraffin, starch, tragacanth, cellulose derivatives, polyethylene glycol, silicone, bentonite, silica, talc, zinc oxide, or the like.

If the formulation of the present invention is a powder or spray, a carrier ingredient used may be lactose, talc, silica, aluminum hydroxide, calcium silicate, or polyamide powder. Particularly, if the formulation is a spray, it may further contain a propellant such as chlorofluorohydrocarbon, propane/butane, or dimethyl ether.

In one embodiment of the present invention, the cosmetic composition may contain a thickener. Examples of a thickener that may be contained in the cosmetic composition include methyl cellulose, carboxyl methyl cellulose, carboxyl methyl hydroxy guanine, hydroxy methyl cellulose, hydroxyethyl cellulose, a carboxyl vinyl polymer, polyquaternium, cetearyl alcohol, stearic acid, carrageenan, and the like. Preferably, the thickener may be one or more of carboxyl methyl cellulose, a carboxyl vinyl polymer, and polyquaternium. More preferably, the thickener may be a carboxyl vinyl polymer.

In one embodiment of the present invention, the cosmetic composition may contain a variety of suitable bases and additives as needed, and the types and amounts of these ingredients may be easily selected by the inventor. If necessary, the cosmetic composition may further contain acceptable additives, for example, ingredients such as a preservative, a pigment, additives, and the like, which are commonly used in the art.

The preservative may specifically be phenoxyethanol or 1,2-hexanediol, and the fragrance may be an artificial fragrance.

In one embodiment of the present invention, the cosmetic composition may contain a composition selected from the group consisting of a water-soluble vitamin, an oil-soluble vitamin, a polymeric peptide, a polymeric polysaccharide, a sphingolipid, and a seaweed extract. Other ingredients that may be added include fats and oils, humectants, emollients, surfactants, organic and inorganic pigments, organic powders, ultraviolet absorbers, preservatives, disinfectants, antioxidants, plant extracts, pH adjusters, alcohols, pigments, fragrances, blood circulation accelerators, cooling agents, anhidrotics, purified water, and the like.

In addition, other ingredients that may be added are not limited thereto. Moreover, any of the above ingredients may be added within the range which does not impair the purpose and effect of the present invention.

### MODE FOR INVENTION

Hereinafter, the present invention will be described in detail by way of examples to assist in the understanding of the present invention. However, the following examples are intended only to illustrate the content of the present invention, and the scope of the present invention is not limited by the following examples. The examples of the present invention are provided to more completely explain the present invention to those skilled in the art.

### Preparation Example 1: Extraction and Culture of Human Tonsil-Derived Mesenchymal Stem Cells (TMSCs)

Tonsil-derived mesenchymal stem cells (TMSCs) were isolated from human tonsil tissue, obtained by tonsillectomy, in the following manner.

First, human tonsil tissue was washed with phosphate-buffered saline (PBS) (Welgene, Seoul, South Korea) containing 2% antibiotics-antimycotics (Gibco, New York, NY, USA). Then, tissue was chopped and digested using 210 U/mL of collagenase type 1 (Gibco, New York, NY, USA) and 4 KU/mL of DNase 1 (Sigma, St. Louis, MO, USA) in low-glucose Dulbecco's modified Eagle's medium (DMEM) (Gibco, New York, NY, USA) at 37° C. for 1 hr and 30 min. The digested product was treated with a stem cell culture medium supplemented with 10% fetal calf serum and 1% antibiotics in DMEM/low glucose, and then filtered through a 40 μm strainer and centrifuged at 1,300 rpm for 3 minutes.

The obtained pellet was washed twice with fresh DMEM. After washing, the obtained cells were cultured in DMEM containing 10% fetal bovine serum (Gibco, New York, NY, USA) and 1% antibiotics-antimycotics at 37° C. and 5% CO₂ atmosphere. The medium was replaced every two days. All mesenchymal stem cells were subcultured through TrypLE express (Gibco, New York, NY, USA) at 5- to 6-day intervals.

FIG. 1a shows the cell morphology of human tonsil stem cells produced in Preparation Example 1, obtained through light microscopy (scale bar=200 μm. LSM 700, ZEISS).

As shown in FIG. 1a, TMSCs had a fibroblast morphology, similar to previously known mesenchymal stem cells.

Meanwhile, TMSCs were characterized by flow cytometry with anti-CD90, anti-CD105, and anti-CD73 antibodies (Biolegend, San Diego, CA, USA).

FIG. 1b shows the expression of surface markers of human tonsil-derived mesenchymal stem cells produced in Preparation Example 1. As shown in FIG. 1b, flow cytometry data showed that TMSCs were positive (>90%) for typical TMSC surface markers, including CD90, CD105, and CD73.

### Preparation Example 2: Selection of CD146-Expressing Cells (CD146+ TMSCs) in Human Tonsil-Derived Mesenchymal Stem Cells

CD146-positive tonsil-derived mesenchymal stem cells were selected from human tonsil-derived mesenchymal stem cells using the human CD146 MicroBead Kit (130-093-596, Miltenyi Biotec, Auburn, USA).

More specifically, the human tonsil-derived mesenchymal stem cells cultured in Preparation Example 1 were washed once with phosphate buffer solution (PBS) and then dissociated by treatment with TrypLE express solution for 3 minutes. DMEM containing 10% fetal bovine serum (Gibco, New York, NY, USA) and 1% antibiotics-antimycotics (Gibco, New York, NY, USA) was added to the dissociated cells, followed by centrifugation at 1,300 rpm for 3 minutes. The supernatant was removed, and the cells were resuspended in PBS and centrifuged under the same conditions. After removing the supernatant, the cell pellet was dissociated by treatment with PBS (60 μL/10⁷ cells) supplemented with 0.5% fetal bovine serum and 2 mM EDTA. Human tonsil-derived stem cells obtained by culturing the cells obtained from the cell pellet were treated with FcR Blocking Reagent (20 μL/10⁷ cells) (BD Biosciences, Franklin Lakes, NJ, USA), treated with CD146 microbeads (20 μL/10⁷ cells), and then incubated under light-shielded conditions at 4° C. for 15 minutes. After incubation, the cells were treated with 1 mL of MACS buffer and centrifuged at 1,300 rpm for 3 minutes, and the supernatant was removed. Thereafter, CD146-positive tonsil-derived mesenchymal stem cells (CD146+ TMSC) and CD146-negative tonsil-derived mesenchymal stem cells (CD146-TMSC) were separated by the MACS separator and LS column (Miltenyi Biotec, Bergisch Gladbach, Germany).

### Preparation Example 3: Production of TMSC-Derived Nanovesicles (TMSC-NV)

For the production of the TMSC-derived nanovesicles, the TMSCs obtained in Preparation Example 1 were dissociated by treatment with TrypLE express solution (Gibco, New York, NY, USA) at 37° C. for 2 minutes. The dissociated cells were suspended in DMEM containing 10% fetal bovine serum (Gibco, New York, NY, USA) and 1% antibiotics-antimycotics (Gibco, New York, NY, USA), and centrifuged at 1,300 rpm for 2 minutes. After removing the supernatant, the obtained cell pellet was washed twice with PBS and resuspended at a density of 1×10⁶ cells/mL in PBS at 10° C.

The resuspended cells were passed sequentially through (porous polycarbonate) filter papers (sandwiched between retainer and extruder) with pore sizes of 10 μm, 5 μm and 0.4 μm, three times per filter paper, using a mini extruder (Avanti polar lipids, Alabaster, AL, USA), thus producing nanovesicles.

### Preparation Example 4: Production of CD146+ TMSC-Derived Nanovesicles (CD146+ TMSC-NVs)

Nanovesicles were produced in the same manner as in Preparation Example 3, except that the CD146+ TMSCs produced in Preparation Example 2 were used instead of the TMSCs produced in Preparation Example 1.

The size and shape of the nanovesicles obtained in Preparation Example 3 and Preparation Example 4 were determined by dynamic light scattering (DLS) and transmission electron microscopy (TEM), respectively.

### 1) Transmission Electron Microscopy (TEM)

Purified nanovesicles were applied to glow-discharged carbon-coated copper grids (Electron Microscopy Sciences, Fort Washington, PA). After the nanovesicles were allowed to absorb onto the grids for 1 hour, the grids were fixed with 4% paraformaldehyde for 10 minutes, washed with droplets of deionized water, and then negatively stained with 2% uranyl acetate (Ted Pella, Redding, CA). Electron micrographs were recorded with a JEM 1011 microscope (JEOL, Tokyo, Japan) at an acceleration voltage of 100 kV.

### 2) Dynamic Light Scattering (DLS)

The size distribution of the nanovesicles was measured using Zetasizer Nano ZS (Malvern Instrument Ltd., Malvern, U.K.).

In addition, the concentration of the nanovesicles was measured using Micro BCA™ Protein Assay Kit (Thermo Fisher Scientific, Waltham, MA, USA).

Meanwhile, to determine the protein expression of the nanovesicles, Western blotting method was used.

### 3) Western Blotting

TMSCs and TMSC-NVs were harvested and lysed in RIPA buffer (Sigma, St. Louis, MO, USA). Lysates were centrifuged at 13,000 rpm for 20 min for removal of cellular debris. The amount of protein in the supernatant was measured using a Micro BCA™ Protein Assay Kit. An amount of 20 μg of total protein was loaded and separated on a 10% SDS-PAGE gel. After loading, separated protein was transferred onto a membrane, which was blocked with 5% BSA solution for 30 min. For immunoblotting, rabbit anti-CD9 (1:2,000), anti-CD63 (1:2,000), and anti-beta actin (1:5,000) primary antibodies (Abcam, Cambridge, UK) were applied at 4° C. overnight.

For chemiluminescence detection of proteins, horseradish peroxidase (HRP)-conjugated goat anti-rabbit IgG (H+L) (1:5,000) secondary antibody (Invitrogen, Carlsbad, CA, USA) was applied at room temperature for 2 hours and Amersham™ ECL Select™ (Thermo Fisher Scientific, Waltham, MA, USA) was used for detection.

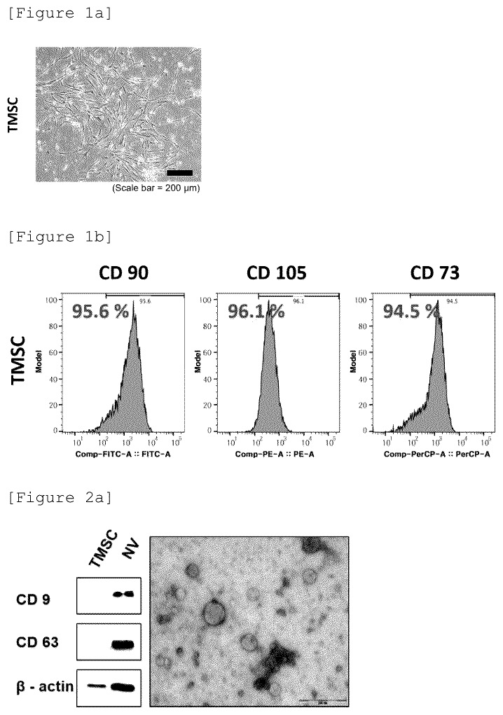

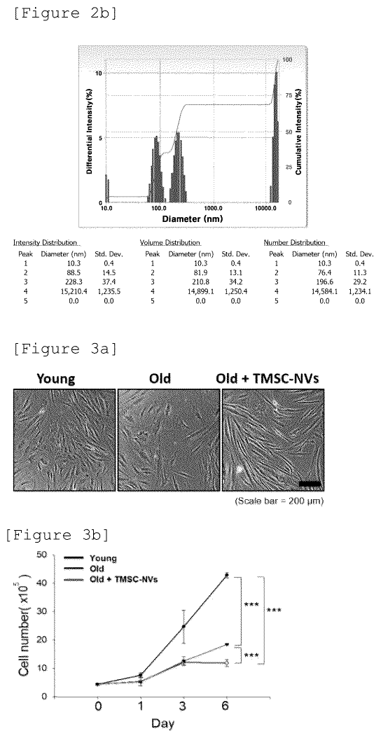

FIG. 2a shows the protein expression and TEM (JEM 1011 microscope (JEOL, Tokyo, Japan) image of TMSC-NVs. Protein level was normalized by β-actin.

FIG. 2b shows the results of dynamic light scattering analysis of TMSC-NVs.

From FIG. 2a, it was confirmed that TMSC-NVs expressed exosome markers such as CD9 and CD63. Meanwhile, TMSC-NVs had a spherical morphology, and from FIG. 2b, it was confirmed that the diameter of TMSC-NVs showed as two peaks (88.5 and 228.3 nm). These results show that the characteristics of TMSC-NVs are similar to those of exosomes.

### Preparation Example 5: Production of Nanovesicles Derived from Adipose-Derived Stem Cells

Nanovesicles were produced in the same manner as Preparation Example 3, except that adipose-derived stem cells were used instead of the TMSCs produced in Preparation Example 1.

### Preparation Example 6: Production of Nanovesicles Derived from Bone Marrow-Derived Stem Cells

Nanovesicles were produced in the same manner as Preparation Example 3, except that bone marrow-derived stem cells were used instead of the TMSCs produced in Preparation Example 1.

### EXPERIMENTAL EXAMPLES

### Cell Culture

Human dermal fibroblasts (HDFs) were purchased from American Type Culture Collection (ATCC, Manassas, VA, USA). Cells were cultured in DMEM containing 10% fetal bovine serum and 1% antibiotics-antimycotics at 37° C. and 5% CO₂ atmosphere. The medium was replaced every two days. Intrinsic replicative senescent cells were produced by repeated passages. Cells at passage 3 and 15 were identified as “young” and “old” states, respectively. Extrinsic senescent cells were treated by UV irradiation with 200 mJ/cm² of UV.

### Experimental Example 1: Comparison of Cell Proliferation Ability

Cell proliferation was measured by Cell Counting Kit-8 assay (CCK-8, Dojindo, Japan). HDFs purchased from ATCC were seeded in a 12-well plate at a density of 5,000 cells/cm² and cultured in stem cell culture medium (DMEM containing 10% fetal bovine serum (Gibco, New York, NY, USA) and 1% antibiotics-antimycotics (Gibco, New York, NY, USA). 24 hours after seeding the HDFs, the HDFs were treated once with the nanovesicles of each of Preparation Example 3 and Preparation Example 4 at a protein concentration of 50 μg/mL and then cultured for 6 days. To compare cell proliferation, each well was treated with a mixture of stem cell culture medium and CCK-8 mixed at a ratio of 1:10. After each well was incubated at 37° C. for 1 hour and 30 minutes, the degree of cell proliferation was compared by measuring the absorbance at a wavelength of 450 nm.

### Experimental Example 2: Immunocytochemistry

To perform immunocytochemistry, the cells of Preparation Example 3 and Preparation Example 4 were fixed with 4% paraformaldehyde for 30 minutes. Then, the fixed cells were permeabilized with 0.05% Triton X-100 (Sigma, St. Louis, MO, USA) for 15 minutes.

After permeabilization, the cells were blocked with 1% bovine serum albumin (BSA) (Sigma, St. Louis, MO, USA) at room temperature for 30 minutes, followed by incubation with anti-vinculin (1:200) at room temperature for 1 hour. After washing with PBS, goat anti-rabbit IgG H&L Alexa Fluor 488 secondary antibodies (1:200) and tetramethylrhodamine-conjugated phalloidin (1:200) were applied for 1 hour in the dark. To confirm ECM production, the cells were fixed, blocked, and incubated with anti-collagen 1 primary antibody (1:200) for 1 hour and incubated with goat anti-rabbit IgG H&L Alexa Fluor 488 (1:200). All antibodies used for immunocytochemistry were purchased from Abcam (Cambridge, MA, USA). Immunocytochemistry was counterstained with DAPI nuclear staining and examined under a ZEISS LSM700 confocal microscope (Zeiss, Oberkochen, Germany).

### Experimental Example 3: Quantitative Real-Time Polymerase Chain Reaction (qPCR)

For quantitative real-time polymerase chain reaction (qPCR), the nanovesicles of Preparation Examples 3 and 4 were treated with senescent fibroblasts for 6 days, and the cells were cultured in 6-well plates.

Each well was washed with 1 mL of PBS, treated with 500 μL of Trizol reagent, and then the sample was collected in a 1.75 mL tube.

Reagent was treated with 200 μL of chloroform and incubated on ice for 10 minutes. The mixture was centrifuged at 13,000 rpm for 15 minutes, and the aqueous supernatant was carefully collected, mixed with an equal amount of isopropanol, and incubated on ice for 10 minutes.

The RNA sample was centrifuged at 13,000 rpm for 15 minutes, the supernatant was removed, and the RNA pellet was collected. The RNA pellet was washed with 75% EtOH and dried.

The transparent RNA pellet was diluted with nuclease-free water, the concentration of RNA was measured using Nanodrop 2000, and cDNA was synthesized from 1 μg of RNA.

cDNA was synthesized using PrimeScript RT Reagent kit (TAKARA, Japan), and AACT value was measured using a Step-One plus qPCR machine (ThermoFisher Scientific, USA).

### Experimental Example 4: Senescence-Associated Beta-Galactosidase Assay (SA-β-Galactosidase Assay)

Senescence-associated beta-galactosidase is an enzyme that catalyzes the hydrolysis of β-galactosides into monosaccharides. It is detectable in senescent cells and tissues at only pH 6.0, not at pH 4.0. As a biomarker of cellular senescence, its activity can be detected using a chromogenic assay using 5-bromo-4-chloro3-indoyl β-D-galactopyranoside (X-gal), which converts to an insoluble blue compound.

Here, SA-β-galactosidase assay was performed using a cellular senescence staining kit (Cell Biolabs, San Diego, CA, USA).

To compare the degree of cell senescence, the SA-β-galactosidase activity of cells was measured using the Cellular Senescence Staining Kit (CBA-230, Cell Biolabs, USA).

More specifically, 6 days after treating the HDFs with the nanovesicles of each of Preparation Examples 3 to 6, the HDFs were washed once with PBS, and then the cells were fixed by treatment with 10% glycerol at room temperature for 5 minutes. After removing the supernatant, the cells were washed three times with PBS and stained using the Cellular Senescence Staining Kit at 37° C. for 14 hours. After reaction, the supernatant was removed, and the cells were washed three times with PBS two times, and then photographed through a microscope and quantitatively analyzed. Quantitative data were measured by documentation of colorization ratio of the senescent cells.

### Experimental Example 5: Examination of Ex Vivo Tissue Regeneration Ability (Immunostaining)

The donated human skin tissue was defatted, washed three times with PBS, and then cut to a size of 1 cm×1 cm. The skin tissue was cultured in a semi-agarose DMEM medium at 37° C. under 5% CO₂. After the human skin tissue being cultured was irradiated with 300 mJ/cm² of ultraviolet B (UVB), 20 μL of CD146+ TMSC-NV was applied to the tissue at concentrations of 50 μg/ml and 100 μg/ml. After 24 hours later, UVB irradiation and the application of CD146+ TMSC-NV were repeated under the same conditions. After repeated treatment, the human skin tissue was transferred to a fresh semi-agarose DMEM medium, subjected to third UVB irradiation and CD146+ TMSC-NV application, and then cultured for 24 hours. Next, the tissue was fixed and immunostained.

Collagen type 1, collagen type 3, involucrin, and filaggrin, which are proteins of the human skin tissue, were stained using immunostaining.

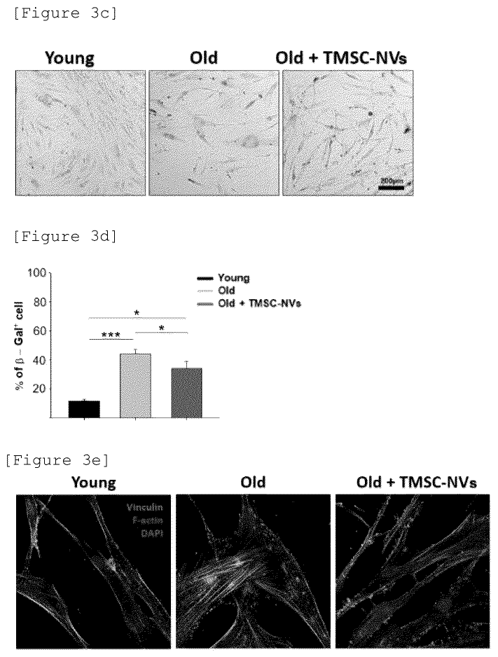

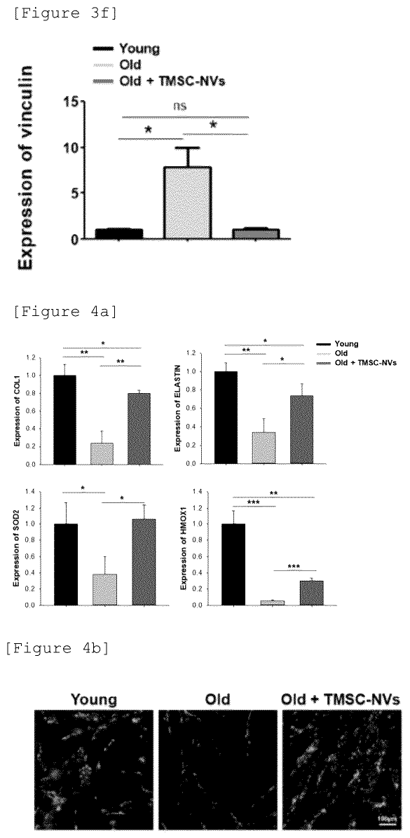

FIG. 3 relates to the regulation of proliferation and senescence by treatment with TMSC-NVs in the passage-associated senescence model. Specifically, FIG. 3a shows morphological changes in HDFs (scale bar=200 μm), FIG. 3b shows the proliferation of passage-associated senescent HDFs after treatment with TMSC-NVs, FIG. 3c shows the results of senescence-associated (SA)-β-galactosidase assay (scale bar=200 μm), FIG. 3d shows the quantitative analysis of SA-β-galactosidase assay, FIG. 3e shows the expression of vinculin in focal adhesion of HDFs, and 3f shows quantitative data of vinculin expression focal in adhesion. Significant differences among groups were determined by one-way ANOVA (ns>0.05, *p<0.05, **p<0.01, ***p<0.001).

As can be seen in FIGS. 3a and 3b, treatment with TMSC-NVs increased the proliferation of senescent HDF cells. SA-β-galactosidase assay was performed to confirm the anti-aging role of TMSC-NVs. As can be seen in FIG. 3c, the results showed that treatment with TMSC-NVs decreased the β-galactosidase activity of old HDFs. Quantitative data from the SA-β-galactosidase assay in FIG. 3d showed that the proportion of senescent cells was reduced by treatment with TMSC-NVs, which supported that treatment with TMSC-NVs decreased the senescence level of HDFs.

In addition, the protein expression of vinculin in focal adhesion and the morphological changes in the actin cytoskeleton after treatment with TMSC-NVs were examined. As shown in FIGS. 3e and 3f, the results of the immunofluorescence analysis show increased commitment of vinculin in focal adhesion in old HDFs, which was decreased by treatment with TMSC-NVs.

These results show that TMSC-NVs increase the proliferation of HDFs and decrease the senescence induced by passages.

In order to confirm the anti-aging properties of TMSC-NVs in terms of molecular biology, the gene expression of the extracellular matrix (ECM) production and senescence-related antioxidant gene after treatment with TMSC-NVs was examined.

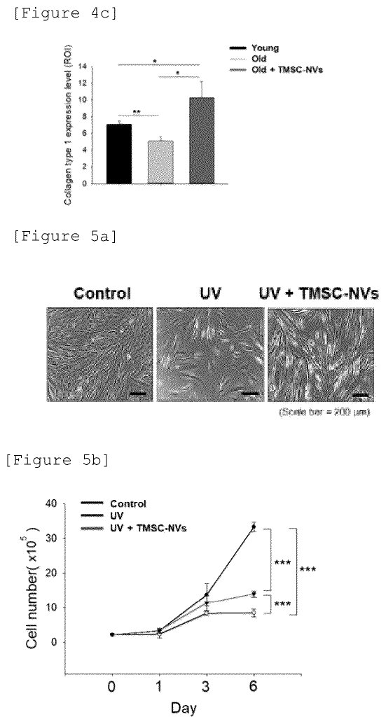

FIG. 4 relates to the regulation of extracellular matrix and antioxidant genes in HDFs by treatment by TMSC-NVs in a passage-associated senescence model. Specifically, FIG. 4a shows the mRNA expression of COL1, ELASTIN, SOD2, and HMOX1 in passage-associated senescent HDFs, FIG. 4b shows the results of immunofluorescence analysis of collagen type 1 in passage-associated senescent aged HDFs, and FIG. 4c shows quantitative data of immunofluorescence analysis. Significant differences among groups were determined by one-way ANOVA (ns>0.05, *p<0.05, **p<0.01, ***p<0.001).

As can be seen in FIG. 4a, the mRNA level of collagen type 1 (COL1) and ELASTIN, which is decreased in old HDFs compared to young HDFs, showed that treatment with TMSC-NVs resulted in upregulated ECM production.

Similarly, the mRNA expression of the antioxidant genes SOD2 and HMOX1 was increased by treatment with TMSC-NVs in old HDFs treated with. In addition, as a result of examining the protein expression of COL1 by immunofluorescence, as shown in FIGS. 4b and 4c, treatment with TMSC-NV resulted in significantly increased ECM production in the passage-associated senescent HDFs. According to these results, it can be seen that treatment with TMSC-NVs results in recovery of the ECM production and senescence-reducible antioxidant gene in senescent cells.

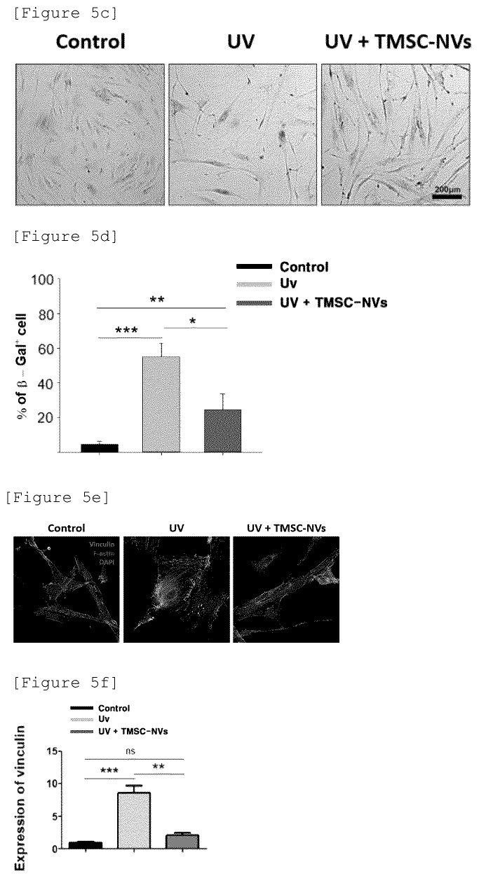

FIG. 5 relates to the regulation of proliferation and senescence by treatment with TMSC-NVs in the UV-induced senescence model. Specifically, FIG. 5a shows morphological changes in HDFs by treatment (scale bar=200 μm), FIG. 5b shows the proliferation of passage-associated senescent HDFs after treatment with TMSC-NVs, FIG. 5c shows the results of senescence-associated (SA)-β-galactosidase assay (scale bar=200 μm), FIG. 5d shows quantitative data of the SA-β-galactosidase assay, FIG. 5e shows the expression of vinculin in focal adhesion of HDFs, and FIG. 5f shows quantitative data of vinculin expression in focal adhesion. Significant differences among groups were determined by one-way ANOVA (ns>0.05, *p<0.05, **p<0.01, ***p<0.001).

As can be seen in FIGS. 5a and 5b, treatment with TMSC-NVs increased the proliferation of UV-induced senescent HDF cells. SA-β-galactosidase assay was performed to confirm the anti-aging role of TMSC-NVs. As can be seen in FIG. 5c, the results showed that treatment with TMSC-NVs decreased the β-galactosidase activity of UV-induced senescent HDFs. Quantitative data from the SA-β-galactosidase assay in FIG. 5d showed that the proportion of senescent cells was reduced by treatment with TMSC-NVs, which supported that treatment with TMSC-NVs decreased the senescence level of HDFs.

In addition, the protein expression of vinculin in focal adhesion and the morphological changes in the actin cytoskeleton after treatment with TMSC-NVs were examined. As shown in FIGS. 5e and 5f, the results of the immunofluorescence analysis show increased commitment of vinculin in focal adhesion in old HDFs, which was decreased by treatment with TMSC-NVs.

These results show that TMSC-NVs increase the proliferation of HDFs and decrease the senescence induced by passages.

To confirm the anti-aging properties of TMSC-NVs in terms of molecular biology, the mRNA expression of the extracellular matrix (ECM) production and senescence-related antioxidant gene in the UV-induced senescence model was examined by qPCR.

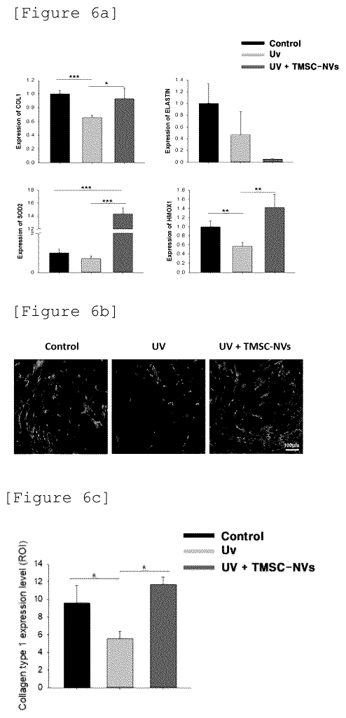

FIG. 6 relates to the regulation of extracellular matrix and antioxidant genes by treatment with TMSC-NVs in the UV-induced senescence model. Specifically, FIG. 6a shows mRNA expression of COL1, ELASTIN, SOD2, and HMOX1 in UV-induced senescent HDFs, FIG. 6b shows s the results of immunofluorescence analysis of collagen type 1 in UV-induced senescent HDFs, and FIG. 6c shows quantitative data of immunofluorescence analysis.

As shown in FIG. 6a, the results of qPCR showed that COL1 and ELASTIN were decreased after UV irradiation and that treatment with TMSC-NVs resulted in significantly increased COL1. However, ELASTIN did not increase. The antioxidant genes SOD2 and HMOX1 were decreased by UV irradiation and increased by treatment with TMSC-NVs. In addition, as shown in FIGS. 6b and 6c, the results of immunofluorescence analysis showed decreased expression of collagen type 1 in UV-induced senescent HDFs, which was increased by treatment with TMSC-NVs. These results indicate that TMSC-NVs increase the ECM production and senescence-reducible antioxidant gene.

In order to confirm the anti-aging properties of CD16+ TMSC-NVs in terms of molecular biology, the gene expression of the extracellular matrix (ECM) production and senescence-related antioxidant genes after treatment with CD146+ TMSC-NVs was examined.

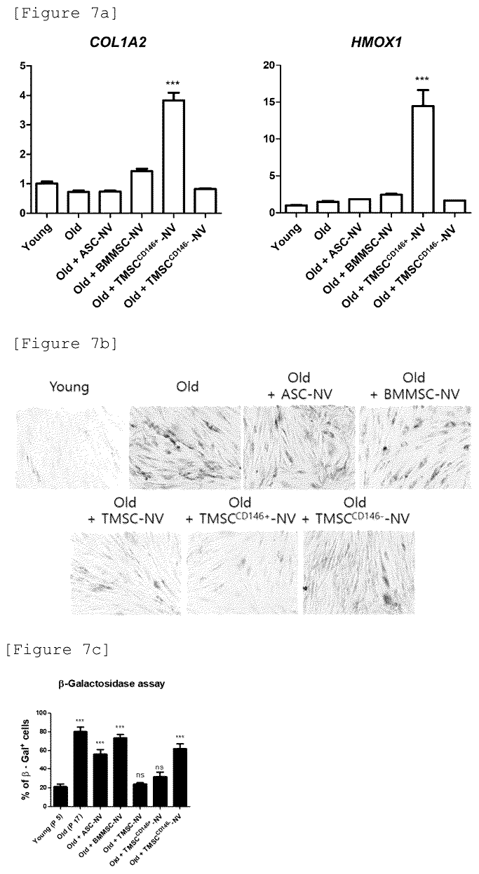

FIG. 7 relates to the regulation of proliferation and senescence by treatment with CD146+ TMSC-NVs in the passage-associated senescence model. Specifically, FIG. 7a shows the mRNA expression of COL1 and HMOX1 in senescent HDFs.

Referring to FIG. 7a, it was confirmed that treatment with CD146+ TMSC-NVs 6 days resulted in an increase in the mRNA expression level of collagen type 1, a skin regeneration-related related marker, and the highest increase in the expression level of HMOX1, an antioxidant-related marker, compared to treatment with nanovesicles derived from adipose-derived stem cells (ASC-NVs) or nanovesicles derived from bone marrow-derived stem cells (BMMSC-NVs).

FIG. 7b is shows the results of senescence-associated β-galactosidase assay (scale bar=200 μm), and FIG. 7c shows the quantitative analysis of SA-β-galactosidase assay,

As can be seen in FIGS. 7b and 7c, it was confirmed that senescent fibroblasts treated with CD146+ TMSC-NVs and senescent fibroblasts treated with TMSC-NVs were stained to a degree almost similar to fibroblasts that are at relatively low passages.

Based on the above results, it was confirmed that CD146+ TMSC-NVs had high anti-aging efficacy and skin regeneration efficacy.

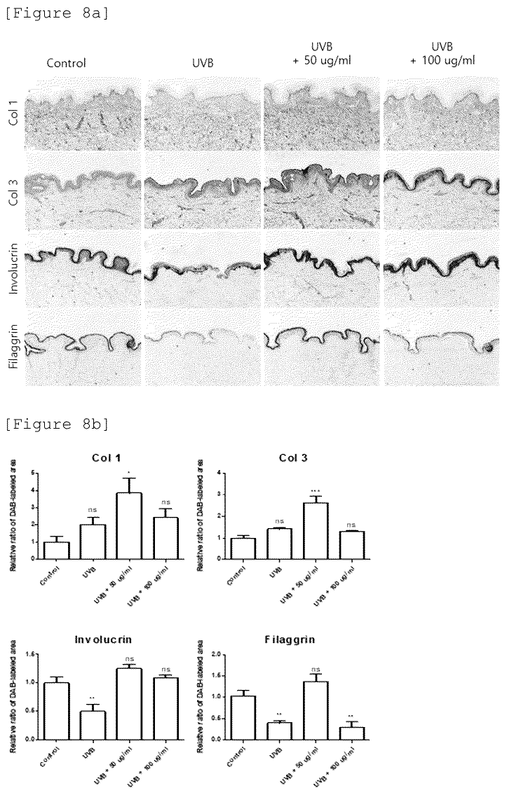

FIGS. 8a and 8b show the results of immunostaining for collagen type 1, collagen type 3, involucrin, and filaggrin of human skin tissue treatment with CD146+ TMSC-NVs in a skin aging model obtained by irradiating human skin tissue with ultraviolet B (UVB).

Based on the above results, it was confirmed that treatment with CD146+ TMSC-NVs repaired UV-induced damage to human skin tissue.

1. A method for skin regeneration and skin anti-aging comprising administering vesicles derived from human tonsil-derived stem cells in a pharmaceutically effective amount to a subject in need thereof.

2. The method for skin regeneration and skin anti-aging of claim 1, wherein the vesicles are one type selected from among extracellular vesicles, microvesicles, and nanovesicles.

3. The method for skin regeneration and skin anti-aging of claim 1, wherein the human tonsil-derived stem cells are human tonsil-derived mesenchymal stem cells.

4. The method for skin regeneration and skin anti-aging of claim 3, wherein the human tonsil-derived stem cells are CD146 positive.

5. The method for skin regeneration and skin anti-aging of claim 2, wherein the nanovesicles have a diameter of 50 nm to 250 nm.

6. The method for skin regeneration and skin anti-aging of claim 1, wherein the vesicles further comprises at least one immune antigen selected from among CD14, CD34, CD45, CD73, CD90, and CD146.

7. The method for skin regeneration and skin anti-aging of claim 1, wherein the human tonsil-derived stem cells are produced by a method comprising steps of:
digesting human tonsil tissue with collagenase type 1 and DNase 1;
filtering and centrifuging the digested product and removing the supernatant to obtain a cell pellet; and
culturing the cells obtained from the cell pellet to obtain human tonsil-derived stem cells.

8. The method for skin regeneration and skin anti-aging of claim 2, wherein the nanovesicles are produced by a method comprising steps of:
suspending subcultured human tonsil-derived mesenchymal stem cells in a culture medium, followed by centrifugation and removing the supernatant; and
resuspending the cell pellet from which the supernatant has been removed, and then passing the cells sequentially through two or more filters with different pore sizes using an extruder.

9. The method for skin regeneration and skin anti-aging of claim 8, wherein the two or more filters with different pore sizes are used in the order from filters with large pore sizes to filters with small pore sizes.

10. The method for skin regeneration and skin anti-aging of claim 8, wherein the two or more filters with different pore sizes are used in the order of filters with pore sizes of 10 μm, 5 μm, and 0.4 μm.

11. The method for skin regeneration and skin anti-aging of claim 7, wherein the method further comprise steps of:
treating the obtained human tonsil-derived stem cells with an FcR blocking reagent, and then treating the cells with CD146 microbeads, followed by incubation;
treating the incubated cells with a magnetic-activated cell sorting (MACS) buffer, followed by centrifugation and removing the supernatant; and
separating CD146-positive and CD146-negative cells by a MACS separator and a column.

12. A cosmetic composition comprising vesicles derived from human tonsil-derived stem cells as an active ingredient.
